(19) United States
(12) Patent Application Publication
(10) Pub. No.: US 2024/0316135 A1
(43) Pub. Date: Sep. 26, 2024

(54) ORALLY CONSUMABLE COMPOSITION WITH PLANT EXTRACT

(71) Applicant: THE COCA-COLA COMPANY, Atlanta, GA (US)

(72) Inventors: Haiyu REN, Atlanta, GA (US); Bin WANG, Atlanta, GA (US)

(21) Appl. No.: 18/573,906

(22) PCT Filed: Jun. 24, 2022
(86) PCT No.: PCT/US2022/034971

§ 371 Date: Dec. 22, 2023

Related U.S. Application Data
(60) Provisional application No. 63/215,285, filed on Jun. 25, 2021.

(51) Int. Cl. A61K 36/534; A61K 9/00; A61K 31/216; A61K 31/522; A61K 36/61; A61P 25/28

(52) U.S. Cl. CPC A61K 36/534; A61K 9/0053; A61K 31/216; A61K 31/522; A61K 36/61; A61P 25/28

(57) ABSTRACT

Provided herein are orally consumable compositions comprising caffeine and a plant extract composition and methods of providing a cognitive benefit by administration of the orally consumable compositions.

This application is being filed on Jun. 24, 2022, as a PCT International Patent application and claims the benefit of and priority to U.S. Provisional patent application Ser. No. 63/215,285, filed Jun. 25, 2021, the entire disclosure of which is incorporated by reference herein in its entirety.

### FIELD

Described herein are orally consumable compositions containing a plant extract composition and/or caffeine, and their use to provide a cognitive benefit.

### SUMMARY

Certain plant extracts and essential oils have previously been studied to investigate cognition enhancing properties, including investigating improvements to cognitive tasks such as memory, attention, accuracy, speed in accomplishing cognitive tasks. Caffeine has similarly also been shown to increase alertness and improve general performance in attention tasks. The cognitive benefits and any synergistic effects derived from the combination of these two compounds with respect to cognitive tasks performance have not previously been investigated. Accordingly, the present disclosure relates to the orally consumable compositions which combine caffeine and a plant extract composition, and methods of providing a cognitive benefit to a human subject through administering the compositions disclosed herein.

The present disclosure relates to orally consumable compositions containing plant extract compositions. More specifically, in one embodiment of the present invention, an orally consumable composition is disclosed comprising:
(i) at least one aqueous extractive(s) from a plant; and/or
(ii) at least one essential oil(s) from the same or different plant; and/or
(iii) caffeine, wherein the caffeine is from a source other than (i) and/or (ii);
wherein (i) and/or (ii) form a plant extract composition; and
wherein the ratio of total caffeine to plant extract composition is from 1:20 to 40:1 by weight (w/w).

In one embodiment, the essential oils are comprised of terpenes, monoterpenes, sesquiterpenes, diterpenes, esters, aldehydes, ketones, alcohols, phenols and oxides, their derivatives and/or a combination thereof.

In embodiments where the plant extract composition includes contains both the essential oil(s) and aqueous extractive(s), the ratio of an essential oil(s) to aqueous extractive(s) is from 1:100 to 1:4.

In some embodiments the plant extract composition is derived from a plant genus selected from a group comprising Salvia, Eucalyptus, Mentha, Thymus, Ocimum, Cinnamomum, Elettaria, Amomum, Pimpinella, Angelica, Zingiber, Piper, Petroselinum, Coriandrum, Foeniculum, Syzygium, Satureia, Camellia, Coffea, Theobroma, Ilex, Paullinia, Euterpe, Ginkgo, Hibiscus, Melissa, Aspalathus, Rosa, Moringa, Magnolia, Olea, Sophora, Crocus, Hypericum, Polygonum, Citrus, Malus, Vitis, Prunus, Litchi, Vaccinium, Lycium, Fragaria, Punica, Ribes, Sambucus, Aronia, Foeniculum, Pimpinella, Beta, Curcuma, Glycine, Juglans, Murraya, Capsicum and combinations thereof.

In embodiments where the plant extract composition is a Salvia extract composition, the Salvia species is selected from S. officinalis, S. lavandulfolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.

In some embodiments, the plant extract composition is selected from a Mentha piperita extract composition, an Eucalyptus globulus composition, a Thymus vulgaris plant extract composition, and a Mentha spicata plant extract composition. In some embodiments, the plant extract composition is a Salvia officinalis extract composition. In further embodiments, the plant extract composition is a Salvia officinalis and Salvia lavandulfolia.

In another embodiment, the present disclosure relates to an orally consumable composition which comprises:
(i) caffeine, wherein a total caffeine content is between 25 mg to 300 mg;
(ii) a plant extract composition comprising at least one aqueous extractive(s) from a plant and/or at least one essential oil(s) from the same or different plant, wherein the plant extract composition is between 1 mg to 500 mg;
wherein the plant extract composition is derived from plant genus Salvia.
   In a further embodiment the plant extract composition can comprise at least one of an essential oil(s) and/or at least one of an aqueous extractive(s), wherein the essential oil(s) is present in an amount from 1 mg to 25 mg of the at least one essential oil(s). In one embodiment the plant extract composition comprises at least one aqueous extract(s), having a rosmarinic acid and/or derivatives thereof in an amount of from 2.5 mg to 100 mg, a carnosol, a carnosic acid, and/or derivatives thereof in an amount of from 0.5 mg to 100 mg, caffeic acid and/or derivatives thereof in an amount of from 2.5 mg to 100 mg, and luteolin and/or derivatives thereof in an amount of from 2.5 mg to 100 mg, in a total amount of up to 500 mg.

In further embodiments, the plant extract composition is a S. officinalis extract composition that comprises rosmarinic acid in an amount of about 3.5 mg to 50 mg, In additional embodiments, the orally consumable composition comprises about 150 mg of the plant extract composition, wherein the plant extract composition is a S. officinalis extract composition that comprises rosmarinic acid in an amount of about 2.5 mg to 100 mg.

In some embodiments, the orally consumable composition is a liquid composition. In some embodiments, the orally consumable composition is in the form of a beverage. In some embodiments, the orally consumable composition is in the form of a carbonated beverage. In some embodiments, the orally consumable composition is in the form of a non-carbonated beverage. In some embodiments, the orally consumable composition is in the form of a liquid concentrate. In some embodiments, the orally consumable the composition is in the form of a solid composition. In some embodiments, the orally consumable composition is in the form of a powder, granule, capsule, tablet, compacted or compressed soluble solid form, food additive, or foodstuff.

In another further embodiment of the present disclosure, provided herein is a method for providing a cognitive benefit to a human subject, comprising orally administering to the subject an orally consumable composition described herein. In another aspect, provided herein is a composition disclosed herein for use in providing a cognitive benefit and/or mood or psychological benefit to a human subject. In another aspect, provided herein is a use of an orally consumable composition described herein for providing a cognitive benefit to a human subject. In some embodiments, the cognitive benefit is one or more selected from improved performance, improved attention, improved accuracy, improved memory, improved alertness, improved tranquility, reduced stress, and reduced mental fatigue. In some embodiments, the method or use further comprises conducting a cognitive assessment of the subject prior to and after the subject has consumed the orally consumable composition.

### BRIEF DESCRIPTION OF THE DRAWINGS

FIG. 1 depicts a COMPASS cognitive assessment comprised of a battery of individual tasks, and illustrates different composite scores that can be derived from results of subsets of the battery of tasks.

FIGS. 2A (Speed of Performance) and 2B (Accuracy of Performance) depict results of cognitive assessments of subjects orally administered compositions as disclosed herein (with different amounts of the plant extract composition), compositions comprising the same amount of caffeine only, and placebo compositions.

FIG. 3 depicts the Mental Fatigue results of cognitive assessments of subjects orally administered compositions as disclosed herein (with different amounts plant extract compositions), compositions comprising the same amount of caffeine only, and placebo compositions.

FIGS. 4A and 4B depict the Alertness results of cognitive assessments of subjects orally administered compositions as disclosed herein (with different amounts of the plant extract composition), compositions comprising the same amount of caffeine only, and placebo compositions. FIG. 4A presents results across all assessments, while FIG. 4B presents results by assessment.

FIG. 5 depicts the Accuracy of Performance results of cognitive assessments of subjects in a secondary study, the subjects of which were orally administered compositions disclosed herein with varying amounts of caffeine and plant extract compositions, caffeine only compositions, and placebo compositions.

FIG. 6 depicts the Accuracy of Attention results of cognitive assessments of subjects in a secondary study, the subjects of which were orally administered compositions disclosed herein, with varying amounts of caffeine and plant extract compositions, caffeine only compositions, and placebo compositions.

FIGS. 7A and 7B depict Serial 3s and Serial 7s results of Cognitive Demand Battery task outcomes of subjects which were orally administered compositions disclosed herein, with varying amounts of caffeine and plant extract compositions, caffeine only compositions, and placebo compositions.

FIGS. 8A and 8B depict RVIP Accuracy and RVIP Speed results of Cognitive Demand Battery task outcomes of subjects which were orally administered compositions disclosed herein, with varying amounts of caffeine and plant extract compositions, caffeine only compositions, and placebo compositions.

### DETAILED DESCRIPTION

The present disclosure relates to orally consumable compositions comprising a plant extract composition and caffeine. As described in more detail below and illustrated in the examples, the research and studies conducted by the inventors surprisingly show that a when a plant extract composition, as described herein, and caffeine are combined, a synergistic effect is produced with respect to one or more cognitive and mood benefits. Thus, methods using the orally consumable compositions as described herein to provide cognitive and/or mood benefits are also disclosed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

Technical and scientific terms used herein have the meanings commonly understood by one of ordinary skill in the art to which the present invention pertains, unless otherwise defined. Reference is made herein to various methodologies known to those of ordinary skill in the art. Suitable materials and/or methods known to those of ordinary skill in the art can be utilized in carrying out the present invention based on the guidance provided herein. However, specific materials and methods are described for illustration. Materials, ingredients and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth. As used herein, “about” will be understood by persons of ordinary skill in the art and will vary to some extent on the context in which it is used. In general, the term “about” is intended to embrace up to plus or minus 10-20% of the particular term. For example, “about 10” is intended to embrace 8-12, and all values therebetween. The recitation of ranges of values herein are intended to serve as shorthand for each separate value falling within the range, unless otherwise indicated, and it should be understood that each separate value is disclosed as if it were individually expressly stated herein.

All methods described herein can be performed in any suitable order unless otherwise indicated herein or otherwise clearly contradicted by context. All examples and exemplary language (including “e.g.” and “such as”) provided herein are intended to better illuminate embodiments of the invention and do not impose limitations on the scope of the invention.

### Compositions

The embodiments disclosed herein relate to orally consumable compositions formulated for oral consumption, comprising caffeine and a plant extract composition as described herein, that are useful, for example, for providing one or more cognitive and/or mood benefit.

As used herein, the term “the plant extract composition” refers to a composition comprising a plant extract, however the term is not limited to plant matter derived through an extraction processes. The term may further refer to compositions of semi-synthetic or synthetic analogs and/or derivatives of plant extracts, derived from synthetic means and not through an extraction process. A plant extract composition may comprise plant matter or synthetic derivatives thereof in raw, purified, unpurified, extracted, or in any other form, singularly or in combination thereof. A plant extract composition may comprise components obtained by subjecting a plant material to any suitable process, such as an extraction process and/or a distillation process. For example, a plant material (such as root, rhizomes, stem, bark, twig, leaves, flowers, fruits, seeds and combinations thereof) may be subject to an aqueous extraction process under elevated temperature, during which volatile components are removed, and one or more volatile components are isolated and reintroduced back into the extractives, to obtain aqueous extractive(s). Additionally or alternatively, a plant material (such as root, rhizomes, stem, bark, twig, leaves, flowers, fruits, seeds and combinations thereof) may be subject to an extraction process, such as an organic extraction process or supercritical fluid extraction process, optionally with subsequent distillation, to obtain organic extractives, such as an essential oil(s). A plant extract composition as described and used herein may comprise one or both of (a) at least one essential oil(s) and/or (b) at least one aqueous extractive(s).

The orally consumable compositions disclosed herein may comprise caffeine. In some embodiments a plant extract composition and caffeine are present, wherein the caffeine is present as an independent caffeine source, which is a source other than the plant extract composition. In other embodiments, a plant extract composition and caffeine are present, however the plant extract composition may itself include a plant species having a caffeine content, in addition to the caffeine present from the independent source in the orally consumable composition. Therefore, it is understood that the compositions disclosed herein can include a “total caffeine” content. In certain embodiments, this total caffeine content represents the sum of caffeine present in the plant extract composition and caffeine present from an independent caffeine source. In other embodiments where the plant extract composition does not include a caffeine content, the total caffeine content of the orally consumable composition would be understood to be comprised solely by the independent caffeine source. In other embodiments, the total caffeine content may be comprised solely of caffeine present in the plant extract composition, where an independent caffeine source is not included in the orally consumable composition.

In one embodiment of the present disclosure an orally consumable composition is disclosed which comprises:
(i) at least one aqueous extractive(s) from a plant; and/or
(ii) at least one essential oil(s) from the same or different plant; and/or
(iii) caffeine, wherein the caffeine is from a source other than (i) and/or (ii);
wherein (i) the at least one aqueous extractive(s) and/or (ii) the at least one essential oil(s) form a plant extract composition;
wherein the ratio of total caffeine to plant extract composition is from 1:20 to 40:1 by weight (w/w).

In one embodiment of the present invention, an orally consumable composition comprises a plant extract composition having one or more aqueous extractive(s), without containing one or more essential oil(s). In other embodiments the plant extract composition may contain one or more essential oil(s) and not contain one or more aqueous extractives(s). In further embodiments, the plant extract composition comprises both, one or more aqueous extractive(s) and one or more essential oil(s).

In one embodiment, the plant extract composition can comprise one or more aqueous extractive(s) and one or more essential oil(s) that are derived from the same plant, and in other embodiments it is envisioned that one or more aqueous extractive(s) and one or more essential oil(s) are derived from different plants.

In the presently disclosed embodiments, the plant extract composition can be derived from a plant genus of Salvia, Eucalyptus, Mentha, Thymus, Ocimum, Cinnamomum, Elettaria, Amomum, Pimpinella, Angelica, Zingiber, Piper, Petroselinum, Coriandrum, Foeniculum, Syzygium, Satureia, Camellia, Coffea, Theobroma, Ilex, Paullinia, Euterpe, Ginkgo, Hibiscus, Melissa, Aspalathus, Rosa, Moringa, Magnolia, Olea, Sophora, Crocus, Hypericum, Polygonum, Citrus, Malus, Vitis, Prunus, Litchi, Vaccinium, Lycium, Fragaria, Punica, Ribes, Sambucus, Aronia, Foeniculum, Pimpinella, Beta, Curcuma, Glycine, Juglans, Murraya, Capsicum and combinations thereof. In some embodiments the plant extract composition incorporates a Salvia species. The Salvia species is selected from S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum and/or any combination thereof.

In further embodiments, the plant extract composition can comprise a Eucalyptus species. The Eucalyptus species is selected from E. globulus, E. cneorifolia, E. dives, E. Dumosa, E. goniocalyx, E. horistes, E. kochii, E. leucoxylon, E. largiflorens, E. oleosa, E. polybractea, E. radiata, E. rossii, E. sideroxylon, E. smithii, E. staigeriana, E. tereticornis, E. viridis and/or combinations thereof.

In additional embodiments the plant extract composition can comprise a Cinnamomum species. The Cinnamomum species is selected from C. burmanni, C. cassia, C. loureirin, C. zeylanicum, C. camphora and/or combinations thereof.

In other embodiments the plant extract composition can comprise a Mentha species. The Mentha species is selected from a group comprising Mentha piperita, M. spicata, M. citrate and combinations thereof. In further embodiments the plant extract composition can comprise a Thymus species. The Thymus species is selected from a group comprising Thymus vulgaris, T. zygis, T serpyllum, T. citriodorus, T. praecox, T. pseudolanuginosus and combinations thereof.

In another embodiment the plant extract composition can comprise an Ocimum species. The Ocimum species is selected from a group comprising Ocimum basilicum, O. citriodorum, O. kilimandscharicum, O. Americanum, O. gratissimum, O. tenuiflorum and combinations thereof.

In a further embodiment the plant extract composition can comprise a Camellia species. The Camellia species is selected from a group comprising Camellia sinensis, C. assamica and combinations thereof.

In an additional embodiment the plant extract composition can comprise a Coffea species. The Coffea species is selected from a group comprising Coffea arabica, C. canephora, C. eugenioides and combinations thereof.

In one embodiment the plant extract composition can comprise a Theobroma species. The Theobroma species is selected from a group comprising Theobroma cacao and combinations thereof.

In a further embodiment the plant extract composition can comprise an Ilex species. The Ilex species is selected from a group comprising Ilex paraguariensis, I. latifolia, I. vomitoria, I. guayusa, I. kudingcha and combinations thereof.

In additional embodiments the plant extract composition can comprise Piper nigrum, Elettaria cardamomum, Amomum subulatum, Pimpinella anisum, Angelica archangelica, Zingiber officinale, Zingiber mioga, Petroselinum crispum, Petroselinum segetu, Coriandrum sativum, Coriandrum tordylium, Foeniculum vulgare, Satureia hortensis, Satureia montana, Syzygium aromaticum and combinations thereof.

In even further embodiments the plant extract composition can comprise a at least one of a plant species selected from a group comprising Paullinia cupana, Euterpe oleracea, Ginkgo biloba, Hibiscus sabdariffa, Melissa officinalis, Aspalathus linearis, Rosa spp., Moringa oleifera, Magnolia officinalis, Olea europaea, Sophora japonica, Crocus sativus, Hypericum perforatum, Polygonum multiflorum, Polygonum cuspidatum, Citrus spp., Malus spp., Vitis spp., Prunus spp., Litchi chinensis, Vaccinium spp., Lycium barbarum, Lycium chinense, Fragaria spp., Punica spp., Ribes spp., Sambucus spp., Aronia melanocarpa, Aronia arbutifolia, Aronia prunifolia, Foeniculum vulgare, Pimpinella anisum, Beta vulgaris, Curcuma longa, Glycine max, Juglans spp., Murraya spp., Capsicum spp. and combinations thereof.

One embodiment of the present disclosure relates to an orally consumable composition comprising (i) caffeine, and (ii) a plant extract composition. The ratio of total caffeine to plant extract composition in the orally consumable composition ranges from 1:20 to 40:1. More specifically, the orally consumable composition may comprise a ratio of (i) caffeine to (ii) plant extract composition in ratios of 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, or any ratio value therebetween.

In some embodiments the plant extract composition comprises at least one of an essential oil(s) and/or at least one of an aqueous extractive(s). As previously disclosed, in some embodiments, the plant extract composition comprises an essential oil(s) but not aqueous extractive(s). In some embodiments, the plant extract composition comprises aqueous extractive(s) but not an essential oil(s). Accordingly, in these embodiments wherein both an essential oil(s) and aqueous extractive(s) are present, the ratio of an essential oil(s) to aqueous extractive(s) ranges from 1:100 to 1:4. More specifically, the ratio of an essential oil(s) to aqueous extractive(s) is 1:100, 1:90, 1:80, 1:70, 1:60, 1:50, 1:40, 1:30, 1:20, 1:10, 1:9, 1:8, 1:7, 1:6, 1:5, or 1:4, or any ratio value therebetween.

### Essential Oil(s)

In an exemplary embodiment, the essential oil(s), when present in the plant extract composition, can comprise terpenes, including monoterpenes, sesquiterpenes, and/or diterpenes, esters, aldehydes, ketones, alcohols, phenols, oxides, and/or their derivatives, and any combination thereof.

Examples of terpenes include, but not limit to, camphene, carene, cymene, limonene, myrcene, phellandrene, pinene, sabinene, terpinene, bisabolene, cadinene, caryophyllene, cedrene, chamazulene, copaene, elemene, farnesene, germacrene D, himachelene, zingiberene.

Examples of esters include, but not limit to, benzyl benzoate, bornyl acetate, geranyl acetate, linalyl acetate, methyl salicylate.

Examples of aldehydes include, but not limit to, cinnamaldehyde, citral, citronellal, geranial, neral.

Examples of ketones include, but not limit to, camphor, carvone, fenchone, jasmone, menthone, pincocamphone. Examples of alcohols include, but not limit to, borneol, citronellol, geraniol, lavandulol, linalool, terpineol, terpinen-4-ol, menthol, nerol, bisabolol, cedrol, farnesol, nerolidol, santalol, zingiberol.

Examples of phenols include, but not limit to, anethole, carvacrol, chavicol, estragole (methyl chavicol), eugenol, thymol.

Examples of oxides include, but not limit to, bisabolol oxide, caryophyllene oxide, 1,8-cineole, linalool oxide, pinene oxide, rose oxide.

According to one exemplary embodiment of the present disclosure, the essential oil(s), when present in a plant extract composition, can comprise borneol (1-5%), myrcene (1-5%), camphene (2-10%), beta-pinene (3-10%), campher (20-55%), 1,8-cineol (20-55%), limonene (3-10%), alpha-pinene (5-15%), of total essential oil(s), in total amount of up to about 25 mg.

In some embodiments, the borneol in the essential oil(s) can be present in an amount of 1, 2, 3, 4, 5%, or any value therebetween, the myrcene can be present in an amount of 1, 2, 3, 4, 5%, or any value therebetween, the camphene can be present in an amount of 2, 3, 4, 5, 6, 7, 8, 9 10%, or any value therebetween, the beta-pinene can be present in an amount 3, 4, 5, 6, 7, 8, 9 10%, or any value therebetween, the campher can be present in an amount of 20, 25, 30, 35, 40, 45, 50, 55%, or any value therebetween. The 1,8-cineol cane be present in an amount of 20, 25, 30, 35, 40, 45, 50, 55%, or any value therebetween, the limonene can be present in an amount of 3, 4, 5, 6, 7, 8, 9 10%, or any value therebetween, the alpha-pinene can be present in an amount 5, 6, 7, 8, 9 10, 11, 12, 13, 14, 15% or any value therebetween.

In the disclosed embodiments, the essential oils(s), when present in the orally consumable composition can comprise plant essential oil derived from the plant root, rhizomes, stem, bark, twig, leaves, flowers, fruits, seeds and combinations thereof.

### Aqueous Extractive(s)

As previously noted the presently disclosed plant extract composition in the orally consumable composition can comprise at least one essential oil(s) and/or at least one aqueous extractive(s). In embodiments wherein at least one aqueous extractive(s) is present, the aqueous extractive can comprise one or more polyphenols.

Accordingly, in some of these embodiments, the one or more polyphenol can include a flavonoid, a phenolic acid, a curcuminoid, a lignan, a stilbene, a phenolic terpene, a glycoside, a glucuronide, a depside and/or combinations thereof.

In some embodiments a flavonoid can comprise a flavanol, a flavone, a flavonol, a flavanone, an isoflavone, an anthocyanidin, a gallate, a glycoside, a polymer and any combination thereof.

In further embodiments a flavanol is selected from a catechin, a epicatechin, a catechin gallate, an epicatechin gallate, a gallocatechin, an epigallocatechin, a gallocatechin gallate, an epigallocatechin gallate and any combinations thereof. A flavone can comprise a luteolin, a chrysoeriol, an apigenin, a tangeritin, a chrysin, a glycoside thereof and any combination thereof. A flavonol can comprise a quercetin, a kaempferol, a myricetin, a rutin, a glycoside thereof and any combination thereof. A flavanone can comprise an eriodictyol, a hesperetin, a narigenin, a glycoside thereof and any combination thereof. An isoflavone can comprise a genistein, a daidzein, a glycoside thereof and any combination thereof. An anthocyanidin can comprise a cyanidin, a delphinidin, a malvidin, a pelargonidin, an aurantinidin, a capensinidin, an europinidin, a hirsutidin, a peonidin, a petunidin, a pulchellidin, a rosinidin, any glycoside thereof and any combination thereof.

In some embodiments where a phenolic acid is present, it can comprise a rosmarinic acid, a vanillic acid, a caffeic acid, a gallic acid, a protocatechuic acid, a salicyclic acid, a ferulic acid, a sinapic acid, a chlorogenic acids, a coumaric acid and any combination thereof.

In another embodiment where a curcuminoid is present it can comprise a bisdemethoxycurcumin, a demethoxycurcumin, a curcumin, and any combination thereof.

In a further embodiment the lignan, if present, is selected from a group comprising a lariciresinol, a pinoresino, a matairesinol, a syrigaresinol, a sesamin, a sesaminol, and any combination thereof.

In an additional embodiment the stilbene if present can be selected from a resveratrol, a pterostilbene, and any combination thereof.

In some embodiments the phenolic terpene is selected from a group comprising a carnosic acid, a rosmanol, a carnosol, and any combination thereof.

In one embodiment, the aqueous extractive(s) can comprise a rosmarinic acid and/or derivatives thereof; carnosol, carnosic acid, and/or derivatives thereof, caffeic acid and/or derivatives thereof and luteolin and/or derivatives thereof, in any permutation or combination. Thus, the aqueous extractive(s), if present, may comprise rosmarinic acid and/or derivatives thereof, or may comprise rosmarinic acid and/or derivatives thereof and carnosol, carnosic acid, and/or derivatives thereof; or may comprise rosmarinic acid and/or derivatives thereof, carnosol, carnosic acid, and/or derivatives thereof, and caffeic acid and/or derivatives thereof, or may comprise rosmarinic acid and/or derivatives thereof, carnosol, carnosic acid, and/or derivatives thereof, caffeic acid and/or derivatives thereof; and luteolin and/or derivatives thereof.

Derivatives of rosmarinic acid:

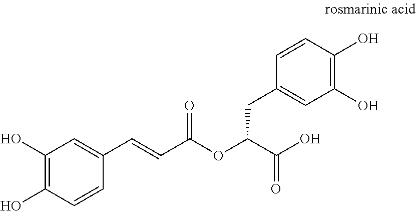 [SMILES: O=C(/C=C/c1ccc(O)c(O)c1)O[C@H](Cc1ccc(O)c(O)c1)C(=O)O]

are described herein, and include lithospermic acid and salvianolic acids, and other derivatives having similar chemical structures.

Derivatives of carnosol and carnosic acid:

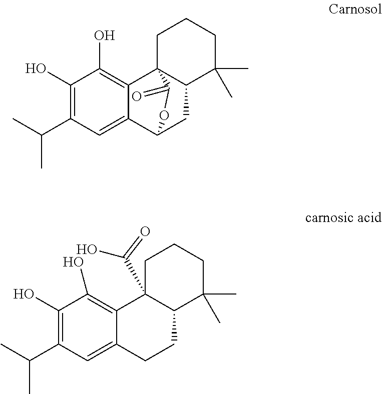

are described herein, and include ferruginol, salvicanol, and pisiferic acid, and other derivatives having similar chemical structures.

Derivatives of caffeic acid:

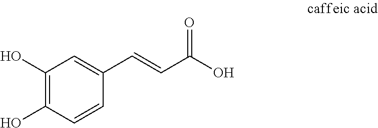 [SMILES: O=C(O)/C=C/c1ccc(O)c(O)c1]

are described herein, and include caffeoylquinic acids, ferulic acid, and feruloylquinic acids, and other derivatives having similar chemical structures.

Derivatives of luteolin:

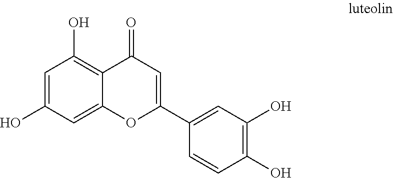 [SMILES: O=c1cc(-c2ccc(O)c(O)c2)oc2cc(O)cc(O)c12]

are described herein, and include luteolin glycosides, apigenin and its glycosides, myricetin and its glucoside, quercetin and its glycosides, kaempferol and its glycosides, salvigenin and its glycosides, diosmetin and its glycosides, baicalin and its glycosides, santin and its glycosides, dinatin and its glycosides, and nepitrin and its glycosides, and other derivatives having similar chemical structures.

In one exemplary embodiment, the plant extract composition comprises at least one aqueous extractive(s) which is comprised of rosmarinic acid and/or derivatives thereof in an amount of from 2.5 mg to 100 mg; carnosol, carnosic acid, and/or derivatives thereof in an amount of from 0.5 mg to 100 mg; caffeic acid and/or derivatives thereof in an amount of from 2.5 mg to 100 mg, and luteolin and/or derivatives thereof in an amount of from 2.5 mg to 100 mg, in a total amount of up to 500 mg.

In another embodiment, the aqueous extractive(s) is derived from a fresh brewed tea composition which comprises catechin, epicatechin, catechin gallate, epicatechin gallate, gallocatechin, epigallocatechin, gallocatechin gallate, epigallocatechin gallate, and gallic acid.

The aqueous extractive(s), if present, may comprise rosmarinic acid and/or derivatives thereof in an amount of from 2.5 mg to 100 mg; carnosol, carnosic acid, and/or derivatives thereof in an amount of from 0.5 mg to 100 mg; caffeic acid and/or derivatives thereof in an amount of from 2.5 mg to 100 mg, and luteolin and/or derivatives thereof in an amount of from 2.5 mg to 100 mg, in a total amount of aqueous extractive(s) of up to 500 mg.

Thus, rosmarinic acid and/or derivatives thereof may be present in a total amount of 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, or 100 mg, or any value therebetween. Additionally or alternatively, carnosol, carnosic acid, and/or derivatives thereof may be present in a total amount of 0.5, 0.6, 0.7, 0.8, 0.9, 1.0, 1.0, 1.1, 1.2, 1.3, 1.4, 1.5, 1.6, 1.7, 1.8, 1.9, 2.0, 2.1, 2.3, 2.4, 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, or 100 mg, or any value therebetween. Additionally or alternatively, caffeic acid and/or derivatives thereof may be present in a total amount of 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, or 100 mg, or any value therebetween. Additionally or alternatively, luteolin and/or derivatives thereof may be present in a total amount of 2.5, 5, 7.5, 10, 12.5, 15, 17.5, 20, 22.5, 25, 27.5, 30, 32.5, 35, 37.5, 40, 42.5, 45, 47.5, 50, 55, 60, 65, 70, 75, 80, 85, 90, 95, or 100 mg, or any value therebetween.

In one embodiment the orally consumable composition may comprise (i) from 25 mg to 300 mg caffeine, and (ii) from 1 mg to 500 mg of the plant extract composition, wherein the ratio of caffeine to plant extract composition in the orally consumable composition is from 1:20 to 40:1.

Thus, the orally consumable composition may comprise about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg, or any value therebetween, of caffeine. In some embodiments, the orally consumable composition comprises about 75 mg caffeine. In additional embodiments the orally consumable composition comprises about 60 mg caffeine.

The orally consumable composition may comprise about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 mg, or any value therebetween, of the plant extract composition.

In accordance with further embodiments, there are provided orally consumable compositions, which comprise,
(i) caffeine, wherein a total caffeine content is between 25 mg to 300 mg;
(ii) a plant extract composition comprising at least one aqueous extractive(s) from a plant and/or at least one essential oil(s) from the same or different plant,
wherein the plant extract composition is between 1 mg to 500 mg;
wherein the plant extract composition is derived from plant genus Salvia.

In further embodiments the plant extract composition comprises at least one of an essential oil(s) and/or at least one of an aqueous extractive(s), wherein the essential oil(s) are present from 1 mg to 25 mg.

In even further embodiments, the an essential oil(s), if present, comprise borneol (1-5%), myrcene (1-5%), camphene (2-10%), beta-pinene (3-10%), campher (20-55%), 1,8-cineol (20-55%), limonene (3-10%), alpha-pinene (5-15%), in a total amount of up to 25 mg.

In a further embodiment, the plant extract composition can further include an the aqueous extractive(s), comprising rosmarinic acid and/or derivatives thereof in an amount of from 2.5 mg to 100 mg; carnosol, carnosic acid, and/or derivatives thereof in an amount of from 0.5 mg to 100 mg; caffeic acid and/or derivatives thereof in an amount of from 2.5 mg to 100 mg, and luteolin and/or derivatives thereof in an amount of from 2.5 mg to 100 mg, in a total amount of up to 500 mg. If both an essential oil(s) and aqueous extractive(s) are present, the ratio of an essential oil(s) to aqueous extractive(s) is from 1:100 to 1:4.

In some embodiments the plant extract composition is derived from Salvia officinalis and Salvia lavandulifolia. In an additional embodiment plant extract composition is a S. officinalis extract composition that comprises rosmarinic acid in an amount of about 3.5 mg to 50 mg.

In another embodiment the orally consumable composition comprises about 150 mg of the plant extract composition, and the plant extract composition is a S. officinalis extract that comprises rosmarinic acid in an amount of about 7.8 mg, and total polyphenol (gallic acid equivalence) in an amount of about 27 mg.

In further embodiment, the orally consumable composition comprises about 250 mg of the plant extract composition, and the plant extract composition is a S. officinalis extract composition that comprises rosmarinic acid in an amount of about 12.9 mg, and total polyphenol (gallic acid equivalence) in an amount of about 45 mg.

In another embodiment, the orally consumable composition comprises about 150 mg of the plant extract composition, and the plant extract composition is a S. officinalis extract composition that comprises rosmarinic acid in an amount of about 7.8 mg, tea catechins in an amount of about 200 mg, and total polyphenol (gallic acid equivalence) in an amount of about 500 mg. The caffeine content is about 60 mg.

In this embodiment, the aqueous extractive(s) in the plant extract composition is derived from a fresh brewed tea composition which comprises catechin, epicatechin, catechin gallate, epicatechin gallate, gallocatechin, epigallocatechin, gallocatechin gallate, epigallocatechin gallate, and gallic acid.

In some embodiments the orally consumable composition comprises a total caffeine content of about 25 to 300 mg. In other embodiments the total caffeine content is about 30-290 mg, 40-280 mg, 50-270 mg, 60-260 mg, 70-250 mg, 80-240 mg, 90-230 mg, 100-220 mg, 110-210 mg, 120-200 mg, 130-190 mg, 140-180 mg, 150-170 mg. In other embodiments the total caffeine content is about 75 mg of caffeine. In further embodiments the total caffeine content is about 60 mg of caffeine.

The plant extract composition may be a Salvia plant extract composition. The Salvia may be S. officinalis, S. lavandulfolia, S. rosmarinus (Rosmarinus officinalis), S. bowleyana, S. cavaleriei, S. chinensis, S. flava, S. prionitis, S. sonchifolia, S. yunnanensis, S. miltiorrhiza, S. multicaulis, or S. divinorum. Thus, in some embodiments, the plant extract composition is a S. officinalis plant extract composition.

In some embodiments, the plant extract composition is a S. lavandubfolia plant extract composition. In some embodiments, the plant extract composition is a S. rosmarinus (Rosmarinus officinalis) plant extract composition. In some embodiments, the plant extract composition is a S. bowleyana plant extract composition. In some embodiments, the plant extract composition is a S. cavaleriei plant extract composition. In some embodiments, the plant extract composition is a S. chinensis plant extract composition.

In some embodiments, the plant extract composition is a S. flava plant extract composition. In some embodiments, the plant extract composition is a S. prionitis plant extract composition. In some embodiments, the plant extract composition is a S. sonchifolia plant extract composition. In some embodiments, the plant extract composition is a S. yunnanensis plant extract composition. In some embodiments, the plant extract composition is a S. miltiorrhiza plant extract composition. In some embodiments, the plant extract composition is a S. multicaulis. In some embodiments, the plant extract composition is a Salvia species is S. divinorum plant extract composition. In some embodiments, the plant extract composition comprises plant extract compositions from a combination of different species of Salvia, such as any combination of two or more of the Salvia species identified above.

Additionally or alternatively, the plant extract composition may be a Mentha piperita plant extract composition. Additionally or alternatively, the plant extract composition may be a Eucalyptus globulus plant extract composition. Additionally or alternatively, the plant extract composition may be a Mentha spicata plant extract composition. Additionally or alternatively, the plant extract composition may be a Thymus vulgaris plant extract composition. In some embodiments, the plant extract composition comprises plant extract compositions from one or more species of Salvia, such as any one or more of the Salvia species identified above, and from one or more of Mentha piperita, Eucalyptus globulus, Thymus vulgaris, and Mentha spicata.

The plant extract composition may be a Salvia officinalis extract composition. In some embodiments, the plant extract composition is a S. officinalis extract composition that comprises rosmarinic acid in an amount of about 3.5 mg to 50 mg.

A plant extract composition as described herein may be obtained commercially or prepared by methods described herein for preparing plant extracts. A plant extract may contain the components outlined above in suitable amounts and ratios thereof to provide a plant extract composition as described herein (e.g., containing borneol (1-5%), myrcene (1-5%), camphene (2-10%), beta-pinene (3-10%), campher (20-55%), 1,8-cineol (20-55%), limonene (3-10%), alpha-pinene (5-15%), in a total amount of up to 25 mg; and/or rosmarinic acid and/or derivatives thereof in an amount of from 2.5 mg to 100 mg; carnosol, carnosic acid, and/or derivatives thereof in an amount of from 0.5 mg to 100 mg; caffeic acid and/or derivatives thereof in an amount of from 2.5 mg to 100 mg, and luteolin and/or derivatives thereof in an amount of from 2.5 mg to 100 mg, in a total amount of up to 500 mg). Alternatively, a plant extract composition as described herein may be obtained by supplementing a plant extract with one or more components as required to achieve the listed components and amounts thereof.

In some embodiments, the orally consumable composition comprises about 150 mg of the plant extract composition. In specific embodiments, the orally consumable composition comprises about 150 mg of the plant extract composition, wherein the plant extract composition is a S. officinalis extract composition that comprises rosmarinic acid in an amount of about 7.8 mg, and total polyphenol (gallic acid equivalence) in an amount of about 27 mg. In specific embodiments, the orally consumable composition comprises about 150 mg of the plant extract composition, wherein the plant extract composition is a S. officinalis extract composition that comprises rosmarinic acid in an amount of about 5.2%, w/w.

In other embodiments, the orally consumable composition comprises about 250 mg of the plant extract composition. In specific embodiments, the orally consumable composition comprises about 250 mg of the plant extract composition, wherein the plant extract composition is a S. officinalis extract composition that comprises rosmarinic acid in an amount of about 12.9 mg, and a total polyphenol (gallic acid equivalents) in an amount of about 45 mg. In specific embodiments, the orally consumable composition comprises about 250 mg of the plant extract composition, wherein the plant extract composition is a S. officinalis extract composition that comprises rosmarinic acid in an amount of about 5.2%, w/w.

The orally consumable compositions disclosed herein may be liquid compositions or solid compositions. In some embodiments, the composition is a liquid composition in the form of a beverage. In some embodiments, the liquid composition is in the form of a carbonated beverage, such as a soft drink, sparkling beverage, or malt beverage. In some embodiments, the liquid composition is in the form of a non-carbonated beverage. In some embodiments, the non-carbonated beverage is a tea. In some embodiments, the non-carbonated beverage is a juice or juice drink (e.g., a fruit and/or vegetable juice or juice drink). In some embodiments, the non-carbonated beverage is a sports drink, energy drink, or protein drink, dairy drink, herbal drink, or tea-based drink. In some embodiments, the beverage is a coffee, cocoa drink, or milk, or other non-dairy alternative milk drink. In some embodiments, the liquid composition is in the form of a liquid concentrate. In some embodiments, the composition is a solid composition in the form of a powder, granules, capsule, tablet, compacted or compressed soluble solid (e.g., cube comprising compacted or compressed powder or granules), food additive or foodstuff.

The orally consumable compositions disclosed herein may be packaged in single serving units, such as single serving units that each contain an amount of the orally consumable composition that provides (i) from 25 mg to 300 mg caffeine, and (ii) from 1 to 500 mg of the plant extract composition. Alternatively, orally consumable compositions disclosed herein may be packaged in bulk form, with each package containing more than one single serving, such as 2, 3, 4, 5, 6, 10, 12, or more, single servings, or any amount of the composition. For example, liquid embodiments may be packaged in bottles, vials or the like. Solid embodiments may be prepared as capsules or tablets, each containing a single serving or a fraction thereof (such as a 14 serving in each capsule or tablet). Alternatively, solid compositions may be prepared as powders or granules packaged in single serving units or bulk forms, such as single serving packets (e.g., stick packs or sachets) or bulk packages (such as packets, bags, jars, tubs, etc.) containing more than one single serving, such as 2, 3, 4, 5, 6, 10, 12, or more, single servings, or any amount of the composition.

### Methods

As noted above, the present inventors surprisingly found that the orally consumable compositions disclosed herein provide one or more cognitive benefits. Thus, in another aspect, disclosed herein are uses of the orally consumable compositions disclosed herein in methods for providing a cognitive benefit to a human subject, wherein the methods comprise administering to the subject an orally consumable composition as described herein. Also disclosed are orally consumable compositions as described herein for use in providing a cognitive benefit to a human subject.

The cognitive benefit may be one or more selected from improved cognitive performance, improved attention, improved memory, improved alertness, improved tranquility, reduced stress, and reduced mental fatigue. As used herein, “improved cognitive performance” refers to one or both of improved speed of cognitive performance and improved accuracy of cognitive performance. As used herein, “improved attention” refers to one or both of improved speed and improved accuracy of attention. As used herein, “improved memory” refers to one or more of improved episodic memory, improved working memory, and improved speed of memory. As used herein, “improved” or “reduced” refers to an improvement or reduction in a cognitive aspect (e.g., one or more of cognitive performance, improved attention, improved memory, improved alertness, improved tranquility, reduced stress, and reduced mental fatigue) after consumption of an orally consumable composition as disclosed herein, as compared to prior to consumption or as compared to consumption of a similarly formulated placebo, as may be determined by conducting a cognitive assessment of the subject prior to and after consumption of an orally consumable composition as disclosed herein, or by conducting a cognitive assessment of the subject after consumption of an orally consumable composition as disclosed herein and after consumption of a similarly formulated placebo. In some embodiments, an improvement or reduction in a cognitive aspect is relative to consumption of a similarly formulated composition comprising the same amount of caffeine but not the plant extract composition. A suitable cognitive assessment is discussed in more detail below and illustrated in Example 1. A cognitive assessment may be conducted at any suitable time period after oral consumption of an orally consumable composition as described herein, such as within about 300 minutes of oral consumption of the composition, including within about 240 minutes, or within about 180 minutes, or within about 120 minutes, or within about 60 minutes, such as about 60 minutes, about 120 minutes, about 180 minutes, about 240 minutes, or about 300 minutes after oral consumption, or any time therebetween, such as 60-300 minutes after oral consumption, or 60-240 minutes after oral consumption, or 60-180 minutes after oral consumption of the composition, In some embodiments, a cognitive assessment may be assessed about 60 minutes, about 180 minutes or about 300 minutes after oral consumption of an orally consumable composition as described herein.

Thus, disclosed herein are methods for providing a cognitive benefit, such as one or more of improving cognitive performance, improving attention, improving memory, improving alertness, improving tranquility, reducing stress, and reducing mental fatigue, in a human subject, comprising orally administering an orally consumable composition as described herein to a human subject. In another aspect, disclosed herein are uses of an orally consumable composition described herein for providing a cognitive benefit to a human subject, such as one or more of improving cognitive performance, improving attention, improving memory, improving alertness, improving tranquility, reducing stress, and reducing mental fatigue. In another aspect, disclosed herein are orally consumable compositions as described herein, for use in providing a cognitive benefit in a human subject, such as one or more of improving cognitive performance, improving attention, improving memory, improving alertness, improving tranquility, reducing stress, and reducing mental fatigue.

In some aspects, the methods, compositions, or uses are for improving cognitive performance in a human subject. Additionally or alternatively, the methods, compositions, or uses are for improving attention in a human subject. Additionally or alternatively, the methods, compositions, or uses are for improving memory in a human subject. Additionally or alternatively, the methods, compositions, or uses are for improving alertness in a human subject. Additionally or alternatively, the methods, compositions, or uses are for improving tranquility in a human subject. Additionally or alternatively, the methods, compositions, or uses are for reducing stress in a human subject. Additionally or alternatively, the methods, compositions, or uses are for reducing mental fatigue in a human subject.

In accordance with any of the methods, compositions, or uses disclosed herein, the orally consumable composition may be orally administered once daily or more than once daily.

In accordance with any of the methods, compositions, or uses disclosed herein, the methods, compositions, or uses may further include conducting a cognitive assessment of the subject. For example, a cognitive assessment may be conducted prior to administration of an orally consumable composition as described herein. Additionally or alternatively, a cognitive assessment may be conducted about 60 minutes, about 180 minutes or about 300 minutes after oral consumption of an orally consumable composition as described herein.

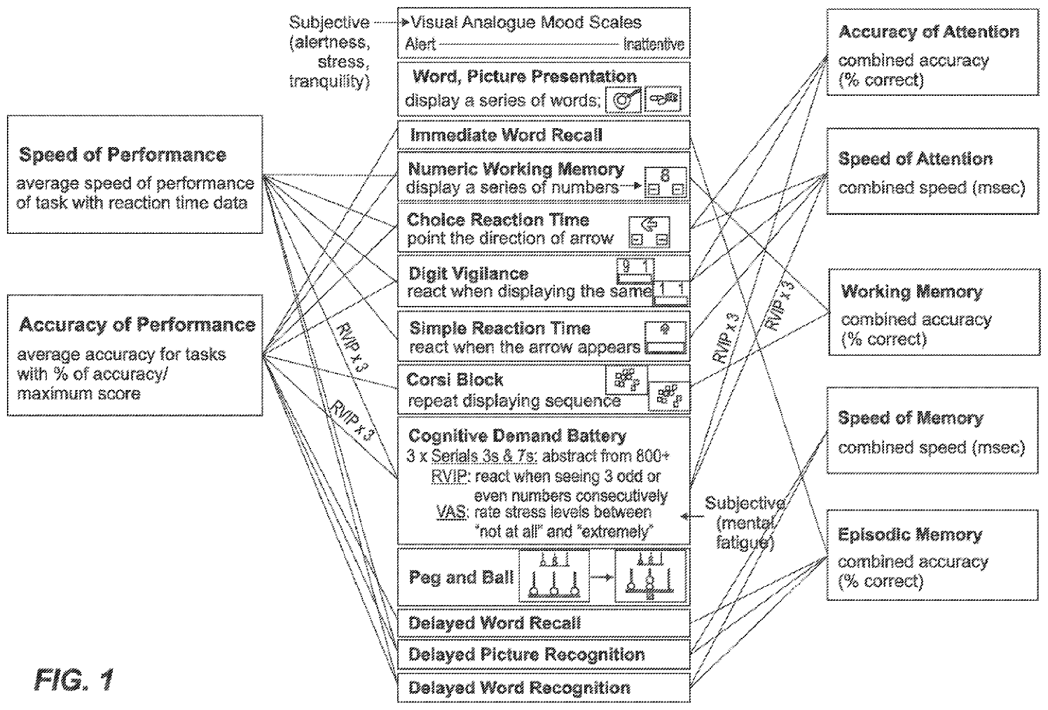

Cognitive Benefit Assessment Cognitive benefit may be assessed by a cognitive assessment, such as a Computerized Mental Performance Assessment System (COMPASS). See, e.g., Wightman et al. Nutrients, 2020, 12(8):2194. This testing system delivers a bespoke tasks, with fully randomized parallel versions of each task delivered at each assessment for each individual. FIG. 1 depicts a battery of individual tasks of a representative COMPASS cognitive assessment, and illustrates different composite scores that can be derived from results of subsets of the battery of tasks. As illustrated, each of the tasks listed in FIG. 1 may be used to produce multiple outcome measures (“scores”). On the right, derivation of “cognitive domain” factor scores is shown; on the left, the tasks are mapped to global speed of performance or accuracy of performance scores. The tasks depicted in FIG. 1 comprised a number of standard tasks that assess aspects of memory (working, episodic, spatial), attention and executive function. Objective measures of visual analogue mood scale can be used to evaluate alertness, stress, and tranquility. For example, three repetitions of the visual analogue scale in the Cognitive Demand Battery (CDB), a sub-sector of COMPASS, can be applied to evaluate mental fatigue. Global cognitive measures can be summarized as accuracy of performance, speed of performance, accuracy of attention, speed of attention, working memory, speed of memory, and episodic memory (FIG. 1). One potential advantage of the COMPASS battery is the possibility of collapsing the task outcomes into “factors” which can be useful to establish if the treatment has a global effect on a given cognitive domain that might escape significance on the component tasks.

For example, a 10-minute Cognitive Demand Battery (CDB) may comprise:
Serial 3s subtraction task (about 2 min): Computerized versions of the serial subtraction tasks can be implemented using tests of about 2-minute duration. Participants are required to count backwards in threes from a given number as quickly and as accurately as possible using the number keys to enter each response. A random starting number between 800 and 999 may be presented on the computer screen, which is cleared by the entry of the first response. The task is scored for number of correct responses and number of errors. In the case of incorrect responses, subsequent responses are scored as positive if correct in relation to the number given as the incorrect response.
Serial 7s subtraction task (about 2 min): This is identical to the serial 3s task with the exception that it involves the serial subtraction of seven.
Rapid Visual Information Processing task (RVIP—about 5 min): The participant is required to monitor a continuous series of digits for targets of three consecutive odd or three consecutive even digits. The digits are presented at the rate of about 100 per minute and the participant responds to detection of a target string by pressing the response button as quickly as possible. The task is continuous and lasts for about 5 minutes, with 8 correct target strings being presented in each minute. The task is scored for percentage of target strings correctly detected, average reaction time for correct detections, and number of false alarms.
Mental fatigue—visual analogue scale: Participants rate their current subjective “‘mental fatigue” state by making a mark on a 100 mm line with the end points labelled “not at all” (left hand end) and “very much so” (right hand end).

A 10-minute Cognitive Demand Battery (CDB) may be completed three times in immediate succession (i.e., for a continuous period of about 30 minutes). COMPASS tasks may comprise:

### Mood/Alertness:

Visual Analogue Mood Scales (VAMS): participants complete 18 visual analogue scales, with each anchored by adjectives describing opposite mood states. The data from the scales is collapsed into three factors previously identified by factor analysis: alertness, stress, and tranquility.

### “Episodic” Long-Term Memory Tasks:

Picture presentation: Fifteen color photographic images of objects are presented sequentially on screen for the participant to remember at the rate of 1 every 3 seconds, with a stimulus duration of about one second.
Word Presentation: A unique set of fifteen words are presented. Words are selected at random from a large bank of words (MRC Psycholinguistic Database) matched for word length, frequency, familiarity and concreteness. Stimulus duration is about one second, as is the inter-stimulus duration.
Immediate and Delayed Word Recall: Participants write down as many of the 15 words that they were presented during the stimulus presentation period immediately after this period and during the delayed recall/recognition period. This task is scored for accuracy only.
Delayed Picture Recognition: A series of pictures is displayed on the screen, one at a time. The number of pictures, the rate at which they are displayed and the inter-stimulus interval can be modified. All target pictures shown during Picture Presentation plus an equal number of decoys are displayed on the screen one at a time. For each stimulus participants select “Yes” or “No” to indicate if they have seen the picture before or not. The task outcomes include accuracy and reaction time.

### Attention:

Digit Vigilance Task: A target digit is randomly selected and constantly displayed to the right of the computer screen. A series of digits is then presented in the center of the screen at the rate of about 80 per minute and the participant is required to press the YES button as quickly as possible every time the digit in the series matches the target digit. The task lasts about 2 minutes and there are 30 stimulus-target matches. Task outcomes are accuracy, mean reaction time and number of false alarms.
Choice Reaction Time: An arrow appears on the screen pointing to the left or to the right. Participants respond with a left or right key press corresponding to the direction of the arrow. There is a randomly varying inter-stimulus interval of between about 1 and about 3 seconds for a total of fifty stimuli. The task outcomes are accuracy and mean reaction time for correct responses.
Simple Reaction Time: Participants respond with a single key press every time a upwards pointing arrow appears on the screen. There is a randomly varying inter-stimulus interval of between about 1 and about 3 seconds for a total of fifty stimuli. Task outcomes are accuracy and mean reaction time.

### Working Memory:

“Sternberg” Numeric Working Memory task: A series of five single digits is displayed on the screen, one at a time. Participants are required to try to memorize these numbers as they appear. Once the series is complete, 30 single digits are displayed one at a time and participants respond to indicate if each number was presented in the previous list or not. The task is repeated three times with different digits. The task outcomes include accuracy and reaction time.
“Corsi Blocks”: Spatial Working Memory Task. Nine blue squares on a black background are displayed on the screen. Some of the blue squares change to red and back to blue again in a sequence. Participants are required to remember this sequence. The task is repeated five times at each level of difficulty with the sequence span increasing from 4 upwards, until the participant can no longer correctly recall the sequences. The task outcome is “span score” and this is calculated as the average of the last 3 correctly completed trials. For example, if the participant correctly responds to all five Level 4 trials and only one Level 5 trial, their span score would be 4.3 [(4+4+5)/3].

Executive Function:
Peg and Ball: Two configurations are shown on the screen. In each there are three colored balls (blue, green, red) on one of 3 pegs. The configuration at the top of the screen is the goal configuration and participants must arrange the balls on the starting configuration (shown in the center of the screen) to match the position of balls in the goal configuration. They must do this in the least number of moves possible. Task outcomes include average thinking time, completion time and errors.

The present invention, thus generally described, will be understood more readily by reference to the following examples, which are provided by way of illustration and are not intended to be limiting of the present invention.

### EXAMPLES

### Example 1. Randomized, Double-Blind, Placebo-Controlled Study

A randomized, double-blind, placebo-controlled study with balanced cross-over design was conducted to assess the effects of acute supplementation with a single administration of a beverage composition as described herein on cognitive function and alertness.

The six beverage compositions tested were:
Placebo (vehicle)
Vehicle with 75 mg caffeine (“Caffeine”)
333 mg sage extract formulated in vehicle (“Sage L”)
545 mg sage extract formulated in vehicle (“Sage H”)
333 mg sage extract formulated in vehicle with 75 mg caffeine (“Sage Combo L”)
545 mg sage extract formulated in vehicle with 75 mg caffeine (“Sage Combo H”)
   The sage (S. officinalis and/or S. lavandulifolia) extract was obtained commercially and contained 50-60% sage extract and 40-50% maltodextrin). Internal analysis confirmed the sage extract contained rosmarinic acid (2.3%, w/w), essential oil (1%, w/w) as formulated with maltodextrin.

The vehicle was a non-commercialized carbonated beverage. All ingredients were food grade and generally recognized as safe (GRAS) by FDA. The ingredients in the vehicle were at the same levels across all beverage compositions.

Participants were randomly allocated to a counterbalancing schedule dictating the order in which they received the six interventions. Participants consumed one of the six interventions within the laboratory during each of their two assessment days (Day 1/Day 2/Day 3/Day 4/Day 5/Day 6), with the order of the interventions counterbalanced across the participants.

The trial utilized the COMPASS cognitive assessment system with Cognitive Demand Battery (CDB) sub-sector of the assessment described above and depicted in FIG. 1 (participants completed the 10-minute CDB battery of tasks three times in immediate succession, for a continuous period of 30 minutes). The order of tasks were as depicted in FIG. 1. The main cognitive/mood assessments took place pre-administration and at 60 minutes, 180 minutes, and 300 minutes post-administration on six separate testing days separated by a minimum of 7 days. The factor scores were calculated by calculating the average of the reaction times (msecs) or % accuracy for all of the tasks that contribute to a given factor (as shown in FIG. 1). Two additional global scores were also calculated using Z scores—Speed of Performance—comprising reaction time data (msec) from all tasks that return this data (with the exception of Peg and Ball); and Accuracy of Performance—comprising % accuracy data from all of the tasks that return this data. Each cognitive function measure was analyzed using mixed model repeated measurement.

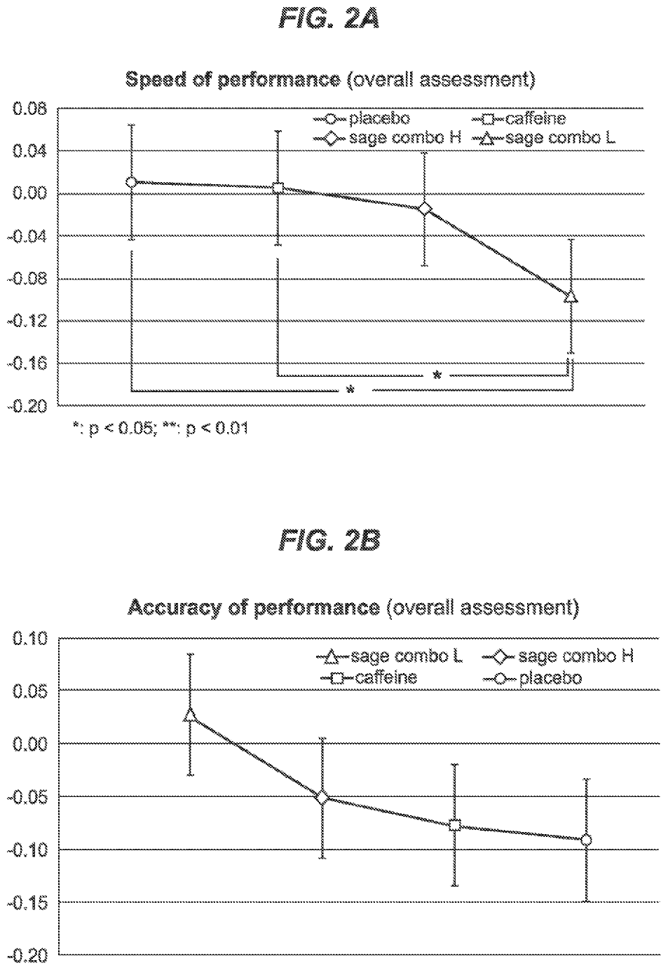

The differences between least squares means for all pairwise comparisons in the treatments were analyzed using either unadjusted or adjusted p value for the main effects. The results showed that the beverage composition formulated with both the sage extract composition (333 mg) and caffeine (75 mg) (Sage Combo L) significantly improved the speed of performance across assessments (60, 180, 300 minutes) compared to placebo (p=0.032) and the beverage composition formulated with caffeine (p=0.039), and improved accuracy of performance also trended towards significance. Although statistical significance was not observed, the results indicate that the improvement of speed of performance was not associated with a reduction in accuracy of performance. See FIGS. 2A and 2B.

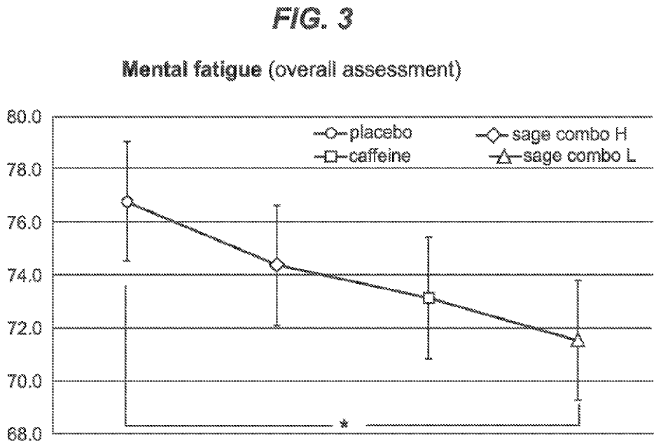

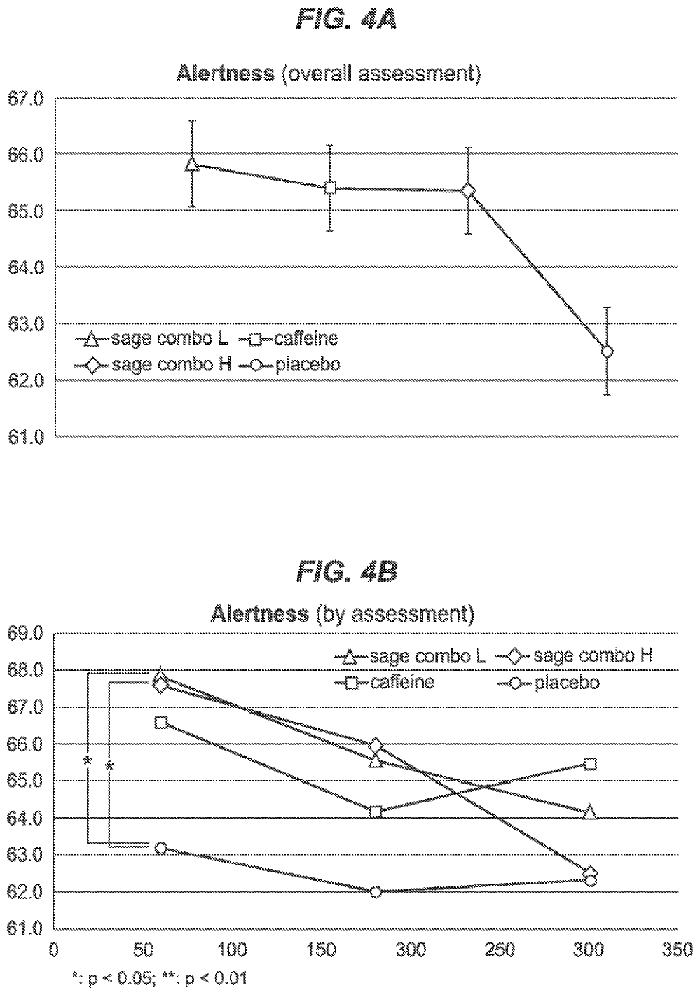

The beverage composition formulated with both the sage extract composition and caffeine (333/75 mg, Sage Combo L) also resulted in significantly reduced mental fatigue across assessments (60, 180, 300 minutes) compared to placebo (p=0.025) (FIG. 3). Additionally, both that sage/caffeine beverage composition (Sage Combo L, p=0.019 and Sage Combo H, p=0.026) improved alertness during the 60 minute assessment as compared to placebo (FIG. 4).

### Example 2

A secondary study was also conducted, which further investigated the effects various compositions disclosed herein, and more particularly of both sage (Salvia officinalis) and caffeine. In the previous investigations these combinations were shown to enhance aspects of cognitive function and elements of mood and psychological state. The aim was to further investigate the synergistic effects of a formulations that combine both sage and caffeine, since such a combination had not previously been assessed for its psychotropic effects in any known studies.

This study adopted a randomized, double-blind, placebo-controlled, balanced cross-over design, in which the acute effects of single doses of sage extract+caffeine (Sage Caffeine Combination), sage extract+tea (Sage in Tea Matrix), caffeinated vehicle and placebo vehicle were assessed on cognitive function and psychological state/mood at 60 min, 180 min and 300 min post-dose.

Participants were randomly allocated to receive one of the following beverages on each of their four study days:
Placebo (flavor vehicle)
75 mg caffeine (in flavor vehicle)
333 mg of Sage (Salvia officinalis and S. lavandulifolia) extract plus 75 mg caffeine (in flavor vehicle) (Sage Caffeine Combination)
333 mg of Sage (Salvia officinalis and S. lavandulifolia) extract in tea containing 60 mg caffeine (Sage in Tea Matrix)

This additional study therefore assessed the effects of single doses of two separate sage containing beverages, one with additional caffeine and one containing tea with a similar level of caffeine, in comparison to a caffeinated beverage and a placebo beverage on multiple aspects of cognitive function and mood/psychological state.

The efficacy of the beverages were assessed with respect to the following measures:
Cognitive function as assessed by global performance measures (Speed of Performance, Accuracy of Performance), cognitive domain factors (Speed of Attention, Accuracy of Attention, Speed of Memory, Episodic Memory, Working Memory) and single task outcomes derived from the COMPASS assessment battery.
Cognitive function and mental fatigue during ‘cognitive demand’ (performance of the Cognitive Demand Battery, comprising repetitions of the Rapid Visual information Processing (RVIP) task, Serial 3s and Serial 7s and a mental fatigue Visual analogue scale)
General Mood/Psychological state (Profile of Mood States, Visual Analogue Mood Scales [VAMS], Stress Visual Analogue Scales ([S-VAS])

The beverages were supplied bottled and identified only by the manufacturers own code. The manufacturers codes were subsequently converted to a site-specific code (A, B, C, D). The order in which participants received the four interventions was counterbalanced across the group via random allocation to a counterbalancing schedule. The drinks were prepared on each day by a disinterested third party according to the counterbalancing schedule.

In the remainder of this disclosure the specific beverages relating to this secondary study will be referred to as ‘placebo’, ‘caffeine’, ‘Sage Tea Matrix’ and ‘Sage Caffeine Combo’.

The secondary study revealed that the Sage Caffeine Combo resulted in a strong pattern of overall results, which were associated with benefits that did not reach significance for caffeine alone. The Sage Caffeine Combo therefore was shown to result in significantly better performance than caffeine alone, particularly in two tasks that load heavily on executive function. Furthermore, the Sage Tea Matrix results in some mood benefits that were not seen after caffeine alone (including confusion, anger, and friendliness factors). The overall results therefore provide evidence of independent or modulatory effects of the non-caffeine components of the sage interventions.

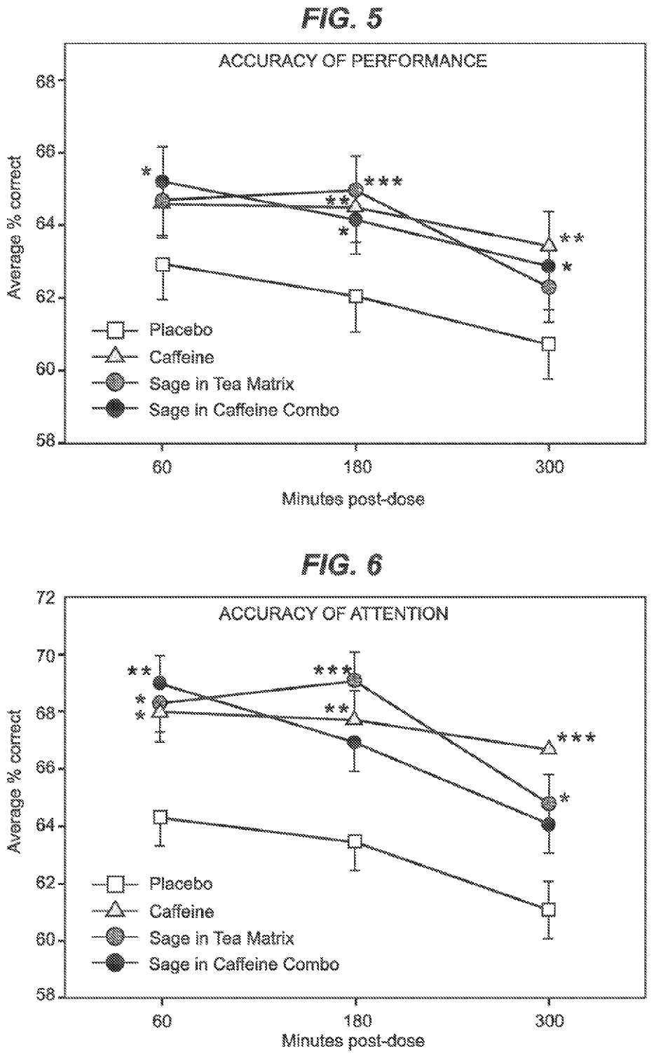

As can be seen in to FIG. 5, the results of the for effects of Sage Caffeine Combo, Sage Tea Matrix, caffeine and placebo drinks on the global ‘Accuracy of Performance’ measure derived from the COMPASS cognitive tasks, can be seen. The graph shows time course data from each post-dose assessment. Asterisks represent significant Bonferroni comparison between the active intervention and placebo at that time point. *, p<0.05; **, p<0.01, ***, p<0.001. The analysis of the global speed and accuracy outcomes showed that there was a significant main effect of the intervention on the ‘Accuracy of Performance’ measure (representing data from the eleven tasks that return % accuracy/maximum score data) [F (3, 482)=12.3, p<0.001]. Reference to the planned comparisons of data averaged across assessments showed that all three active drinks were associated with significantly better (all P<0.001) accuracy post-dose. The comparisons of mean data from each assessment timepoint showed that whilst only Sage Caffeine Combo was associated with improvements at 60 minutes post-dose (p<0.05), all three drinks resulted in improved performance at 180 minutes (caffeine p<0.01, Sage Caffeine Combo p<0.05, Sage Tea Matrix p<0.001) and both caffeine (p<0.01) and Sage Tea Matrix (p<0.05) enhanced accuracy at 300 minutes post-dose.

In FIG. 6, results are disclosed for ‘Accuracy of Attention” factors as impacted by the effects of Sage Caffeine Combo, Sage Tea Matrix, caffeine and placebo drinks. These cognitive factors were derived from the COMPASS cognitive task outcomes. The graph shows time course data from each post-dose assessment for those measures that evinced such effects (i.e. an absence of a line graph denotes that were no significant effects on these comparisons for the outcome). Asterisks represent significant Bonferroni comparison between the intervention and placebo at that time point. *, p<0.05; **, p<0.01, ***, p<0.001. The data charted in the graph shows that in line with overall improvements in accuracy, there was also a main effect on the Accuracy of Attention factor (combining % accuracy data from 5 attention tasks) [F (3, 240)=9.0, p<0.001]. Reference to the planned comparisons showed that all three drinks resulted in improved performance across the post-dose testing day in comparison to placebo (caffeine, p<0.001; Sage Tea Matrix, p<0001; Sage Caffeine Combo, p<0.01). Comparisons of data from individual assessments showed that whereas Sage Caffeine Combo was associated with significant improvements over placebo during the 60 min assessment (p<0.01), both caffeine and Sage Tea Matrix were associated with improvements during all three assessments (caffeine, 60 min—p<0.05, 180 min—p<0.01, 300 min—p<0.001: Sage Tea Matrix, 60 min—p<0.05, 180 min—p<0.001, 300 min—p<0.05).

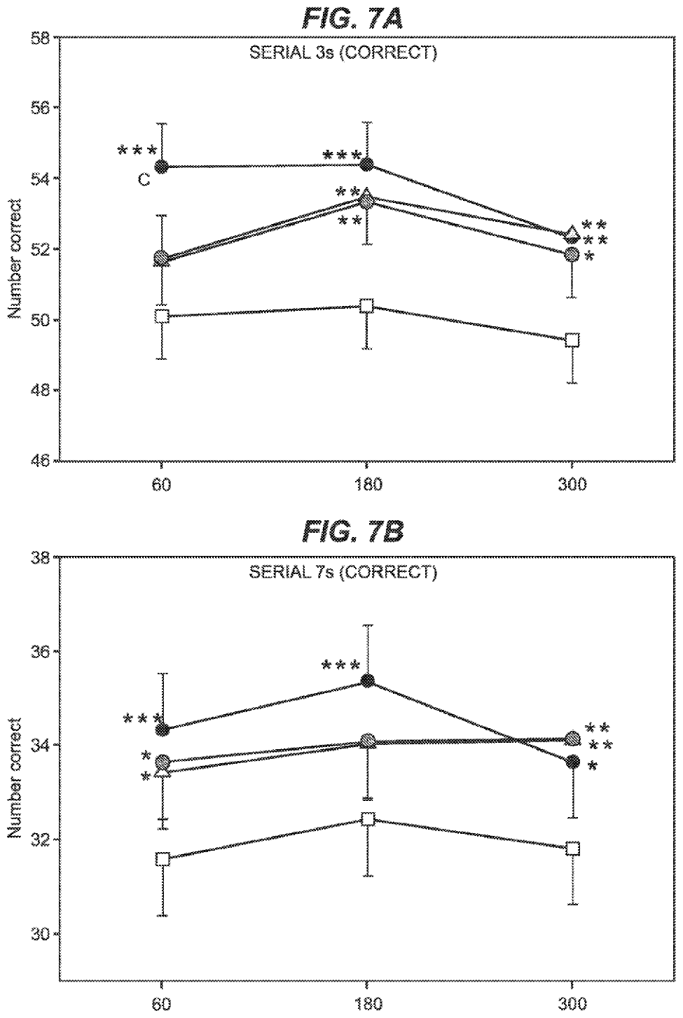

As shown in FIGS. 7A and 7B, there was a main effect on the total number of correct Serial 3s [F(3, 1600)=17, p<0.001] and Serial 7s [F(3, 1602)=14.3, p<0.001] reflecting improved performance in comparison to placebo for all three active drinks across the post-dose assessments (p<0.001 in all cases). In the case of both Serial 3s and 7s the clearest pattern of benefits was seen following Sage Caffeine Combo, which resulted in the strongest improvements at 60 min and 180 min post-dose in comparison to placebo (p<0.001 in all cases) with these effects attenuating at 300 min post-dose (Serial 3s p<0.01; Serial 7s p<0.05). Sage Caffeine Combo also saw a significant improvement in comparison to caffeine at 60 min post-dose on this Serial 3s outcome (p<0.05). Both Sage Tea Matrix and caffeine were also associated with benefits in the total number of subtractions. On the Serial 3s task these were seen at 180 min (both p<0.01) and 300 min (caffeine, p<0.01; Sage Tea Matrix p<0.05) post-dose and on the Serial 7s at 60 min (both p<0.05) and 300 min (both p<0.01) post-dose.

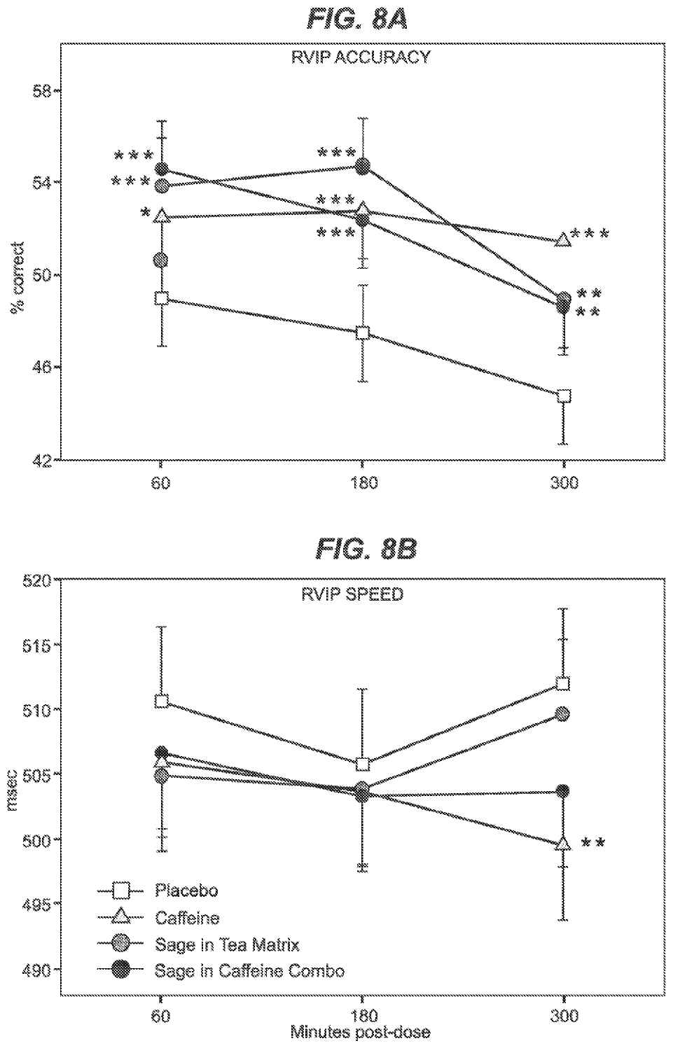

FIGS. 8A and 8B show that there was also a main effect with regards the accuracy of performing the Rapid Visual Information Processing (RVIP) task [F (3, 1499)=28.3. p<0.001], with all three drinks outperforming placebo across the post-dose period (all p<0.001). The results are depicted in FIGS. 8A and 8B. Reference to the planned comparisons at each time point showed that all three drinks resulted in significant benefits at 60-min (caffeine, p<0.05; Sage Caffeine Combo/Sage Tea Matrix, p<0.001), 180 min (all drinks p<0.001) and 300 min (caffeine, p<0.001, Sage Caffeine Combo/Sage Tea Matrix, p<0.01) post-dose. There was also a much less pronounced main effect on the speed of performing the RVIP [F (3, 1500)=3.54, p<0.05], with significantly faster performance in comparison to placebo seen across assessments following caffeine (p<0.01), with a trend towards the same effect for Sage Tea Matrix (P<0.1).

These performance benefits were matched by reduced ratings of mental fatigue during the CDB (main effect: [F (3, 1608)=23.5, p<0.001]) for all three active interventions in comparison to placebo (all, p<0.001). With reference to the individual assessments, mental fatigue was also reduced by all three interventions at 60 min (caffeine, p<0.01, Sage Caffeine Combo/Sage Tea Matrix, p<0.001), 180 min (caffeine p<0.05, Sage Caffeine Combo/Sage Tea Matrix, p<0.001) and 300 min (caffeine, p<0.001, Sage Caffeine Combo, p<0.01, Sage Tea Matrix, p<0.05) post-dose.

Overall, these results provide evidence that orally consumable compositions as described herein comprising a plant extract compositions, such as a sage plant extract composition, and caffeine provide cognitive and mood/psychological benefits, even as compared to compositions formulated with caffeine alone, and indicate that the sage extract composition and caffeine are acting synergistically. The results also indicate that the cognitive functionalities and benefits generated from sage and caffeine are different.

The following numbered clauses define further example aspects and features of the present disclosure:

1. An orally consumable composition comprising:
(i) at least one aqueous extractive(s) from a plant; and/or
(ii) at least one essential oil(s) from the same or different plant; and/or
(iii) caffeine, wherein the caffeine is from a source other than (i) and/or (ii);
wherein (i) and/or (ii) form a plant extract composition; and wherein the ratio of total caffeine to plant extract composition is from 1:20 to 40:1 by weight (w/w).
   2. The orally consumable composition of clause 1, wherein the at least one essential oil(s), comprise terpenes, monoterpenes, sesquiterpenes, diterpenes, esters, aldehydes, ketones, alcohols, phenols and oxides, and derivatives and/or combinations thereof.
   3. The orally consumable composition of any one of clauses 1-2, wherein if both the essential oil(s) and the at least one aqueous extractive(s) are present, the ratio of the at least one essential oil(s) to the at least one aqueous extractive(s) is from 1:100 to 1:4.
   4. The orally consumable composition of any one of clauses 1-3, wherein the at least one essential oils(s) comprise plant essential oil derived from the plant root, rhizomes, stem, bark, twig, leaves, flowers, fruits, seeds and combinations thereof.
   5. The orally consumable composition of any one of clauses 1-4, wherein the plant extract composition is derived from a plant genus selected from a group comprising Salvia, Eucalyptus, Mentha, Thymus, Ocimum, Cinnamomum, Elettaria, Amomum, Pimpinella, Angelica, Zingiber, Piper, Petroselinum, Coriandrum, Foeniculum, Syzygium, Satureia, Camellia, Coffea, Theobroma, Ilex, Paullinia, Euterpe, Ginkgo, Hibiscus, Melissa, Aspalathus, Rosa, Moringa, Magnolia, Olea, Sophora, Crocus, Hypericum, Polygonum, Citrus, Malus, Vitis, Prunus, Litchi, Vaccinium, Lycium, Fragaria, Punica, Ribes, Sambucus, Aronia, Foeniculum, Pimpinella, Beta, Curcuma, Glycine, Juglans, Murraya, Capsicum and combinations thereof.
   6. The orally consumable composition of any one of clauses 1 to 5, wherein the plant extract composition comprises a Salvia species, wherein the Salvia species is selected from S. officinalis, S. lavandulfolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.
   7. The orally consumable composition of any one of clauses 1 to 6, wherein the plant extract composition comprises a Eucalyptus species, wherein the Eucalyptus species is selected from a group comprising Eucalyptus species is selected from E. globulus, E. cneorifolia, E. dives, E. Dumosa, E. goniocalyx, E. horistes, E. kochii, E. leucoxylon, E. largiflorens, E. oleosa, E. polybractea, E. radiata, E. rossii, E. sideroxylon, E. smithii, E. staigeriana, E. tereticornis, E. viridis and combinations thereof.
   8. The orally consumable composition of any one of clauses 1 to 7, wherein the plant extract composition comprises a Mentha species, wherein the Mentha species is selected from a group comprising Mentha piperita, M. spicata, M. citrate and combinations thereof.
   9. The orally consumable composition of any one of clauses 1 to 8, wherein the plant extract composition comprises a Thymus species, wherein the Thymus species is selected from a group comprising Thymus vulgaris, T. zygis, T. serpyllum, T. citriodorus, T. praecox, T. pseudolanuginosus and combinations thereof.
   10. The orally consumable composition of any one of clauses 1 to 9, wherein the plant extract composition comprises a Ocimum species, wherein the Ocimum species is selected from a group comprising Ocimum basilicum, O. citriodorum, O. kilimandscharicum, O. Americanum, O. gratissimum, O. tenuiflorum and combinations thereof.
   11. The orally consumable composition of any one of clauses 1 to 10, wherein the plant extract composition comprises a Cinnamomum species, wherein the Cinnamomum species is selected from a group comprising C. burmanni, C. cassia, C. loureirin, C. zeylanicum, C. camphora and combinations thereof.
   12. The orally consumable composition of any one of clauses 1 to 11, wherein the plant extract composition comprises a Camellia species, wherein the Camellia species is selected from a group comprising Camellia sinensis, C. assamica and combinations thereof.
   13. The orally consumable composition of any one of clauses 1 to 12, wherein the plant extract composition comprises a Coffea species, wherein the Coffea species is selected from a group comprising Coffea arabica, C. canephora, C. eugenioides and combinations thereof.
   14. The orally consumable composition of any one of clauses 1 to 13, wherein the plant extract composition comprises a Theobroma species, wherein the Theobroma species is selected from a group comprising Theobroma cacao and combinations thereof.
   15. The orally consumable composition of any one of clauses 1 to 14, wherein the plant extract composition comprises a Iex species, wherein the Iex species is selected from a group comprising Ilex paraguariensis, I. latifolia, I. vomitoria, I. guayusa, I. kudingcha and combinations thereof.
   16. The orally consumable composition of any one of clauses 1 to 15, wherein the plant extract composition comprises at least one of a plant species selected from a group comprising Piper nigrum, Elettaria cardamomum, Amomum subulatum, Pimpinella anisum, Angelica archangelica, Zingiber officinale, Zingiber mioga, Petroselinum crispum, Petroselinum segetu, Coriandrum sativum, Coriandrum tordylium, Foeniculum vulgare, Satureia hortensis, Satureia montana, Syzygium aromaticum and combinations thereof.
   17. The orally consumable composition of any one of clauses 1 to 16, wherein the plant extract composition comprises at least one of a plant species selected from a group comprising Paullinia cupana, Euterpe oleracea, Ginkgo biloba, Hibiscus sabdariffa, Melissa officinalis, Aspalathus linearis, Rosa spp., Moringa oleifera, Magnolia officinalis, Olea europaea, Sophora japonica, Crocus sativus, Hypericum perforatum, Polygonum multiflorum, Polygonum cuspidatum, Citrus spp., Malus spp., Vitis spp., Prunus spp., Litchi chinensis, Vaccinium spp., Lycium barbarum, Lycium chinense, Fragaria spp., Punica spp., Ribes spp., Sambucus spp., Aronia melanocarpa, Aronia arbutifolia, Aronia prunifolia, Foeniculum vulgare, Pimpinella anisum, Beta vulgaris, Curcuma longa, Glycine max, Juglans spp., Murraya spp., Capsicum spp. and combinations thereof.
   18. The orally consumable composition of any one of clauses 1-17, wherein the at least one aqueous extractive(s) is derived from at least one of a plant's root, rhizomes, stem, bark, twig, leaves, flowers, fruits, or seeds.
   19. The orally consumable composition of any one of clauses 1-18, wherein the at least one aqueous extractive(s) comprises at least one polyphenol.
   20. The orally consumable composition of clause 19, wherein the polyphenol is selected from a group comprising a flavonoid, a phenolic acid, a curcuminoid, a lignan, a stilbene, a phenolic terpene, a glycoside, a glucuronide, a depside and combination thereof.
   21. The orally consumable composition of clause 20, wherein the flavonoid is selected from a group comprising a flavanol, a flavone, a flavonol, a flavanone, an isoflavone, an anthocyanidin, a gallate, a glycoside, a polymer and any combination thereof.
   22. The orally consumable composition of clause 21, wherein the flavanol is selected from a group comprising a catechin, a epicatechin, a catechin gallate, an epicatechin gallate, a gallocatechin, an epigallocatechin, a gallocatechin gallate, an epigallocatechin gallate and any combinations thereof.
   23. The orally consumable composition of clause 21, wherein the flavone is selected from a group comprising a luteolin, an apigenin, a tangeritin, a chrysin, and any combination thereof.
   24. The orally consumable composition of clause 21, wherein the flavonol is selected from a group comprising a quercetin, a kaempferol, a myricetin, a rutin, and any combination thereof.
   25. The orally consumable composition of clause 21, wherein the flavanone is selected from a group comprising a eriodictyol, a hesperetin, a narigenin, and any combination thereof.
   26. The orally consumable composition of clause 21, wherein the isoflavone is selected from a group comprising a genistein, a daidzein and any combination thereof.
   27. The orally consumable composition of clause 21, wherein the anthocyanidin is selected from a group comprising a cyanidin, a delphinidin, a malvidin, a pelargonidin, an aaurantinidin, a capensinidin, an europinidin, a hirsutidin, a peonidin, a petunidin, a pulchellidin, a rosinidin and any combination thereof.
   28. The orally consumable composition of clause 20, wherein the phenolic acid is selected from a group comprising a rosmarinic acid, a vanillic acid, a caffeic acid, a gallic acid, a protocatechuic acid, a salicyclic acid, a ferulic acid, a sinapic acid, a chlorogenic acids, a coumaric acid and any combination thereof.
   29. The orally consumable composition of clause 20, wherein the curcuminoid is selected from a group comprising a bisdemethoxycurcumin, a demethoxycurcumin, a curcumin, and any combination thereof.
   30. The orally consumable composition of clause 20, wherein the lignan is selected from a group comprising a lariciresinol, a pinoresino, a matairesinol, a syrigaresinol, a sesamin, a sesaminol, and any combination thereof.
   31. The orally consumable composition of clause 20, wherein the stilbene is selected from a group comprising a resveratrol, a pterostilbene, and any combination thereof.
   32. The orally consumable composition of clause 20, wherein the phenolic terpene is selected from a group comprising a carnosic acid, a rosmanol, a carnosol, and any combination thereof.
   33. The orally consumable composition of any one of clause 1-32, wherein the at least one aqueous extractive(s), comprise rosmarinic acid and/or derivatives thereof in an amount of from 2.5 mg to 100 mg; carnosol, carnosic acid, and/or derivatives thereof in an amount of from 0.5 mg to 100 mg; caffeic acid and/or derivatives thereof in an amount of from 2.5 mg to 100 mg, and luteolin and/or derivatives thereof in an amount of from 2.5 mg to 100 mg, in a total amount of up to 500 mg.
   34. An orally consumable composition comprising:
(i) caffeine, wherein a total caffeine content is between 25 mg to 300 mg;
(ii) a plant extract composition comprising at least one aqueous extractive(s) from a plant and/or at least one essential oil(s) from the same or different plant, wherein the plant extract composition is between 1 mg to 500 mg; wherein the plant extract composition is derived from plant genus Salvia.
   35. The orally consumable composition of clause 34, comprising from 1 mg to 25 mg of the at least one essential oil(s).
   36. The orally consumable composition of clause 34 or 35, wherein the at least one aqueous extractive(s) comprises: a rosmarinic acid and/or derivatives thereof in an amount of from 2.5 mg to 100 mg;
a carnosol, a carnosic acid, and/or derivatives thereof in an amount of from 0.5 mg to 100 mg;
caffeic acid and/or derivatives thereof in an amount of from 2.5 mg to 100 mg, and
luteolin and/or derivatives thereof in an amount of from 2.5 mg to 100 mg, in a total amount of up to 500 mg,
   37. The orally consumable composition of any one of clause 1-36, wherein the plant extract composition is a S. officinalis extract composition that comprises rosmarinic acid in an amount of about 3.5 mg to 50 mg.
   38. The orally consumable composition of any one of clause 1-37, comprising about 150 mg of the plant extract composition, wherein the plant extract composition is a S. officinalis extract composition that comprises rosmarinic acid in an amount of about 7.8 mg, and total polyphenol in an amount of about 27 mg.
   39. The orally consumable composition of clause 1-37, comprising about 250 mg of the plant extract composition, wherein the plant extract composition is a S. officinalis extract composition that comprises rosmarinic acid in an amount of about 12.9 mg, and total polyphenol in an amount of about 45 mg.
   40. The orally consumable composition of any one of clauses 1-39, wherein the orally consumable composition comprises about 60-75 mg caffeine.
   41. The orally consumable composition of any one of clauses 28, 33 and 36-40, wherein the rosmarinic acid derivatives comprise lithospermic acid, salvianolic acids, or any combination thereof.
   42. The orally consumable composition of any one of clauses 32, 33 and 36-41 and, wherein the carnosol derivatives or carnosic acid derivatives comprise ferruginol, salvicanol, pisiferic acid, or any combination thereof.
   43. The orally consumable composition of any one of clauses 28, 33 and 36-42, wherein the caffeic acid derivatives comprise caffeoylquinic acids, ferulic acid, feruloylquinic acids, or any combination thereof.
   44. The orally consumable composition of any one of clauses 23, 33, 36-43, wherein the luteolin derivatives comprise luteolin and its glycosides, apigenin and its glycosides, myricetin and its glucoside, quercetin and its glycosides, kaempferol and its glycosides, salvigenin and its glycosides, diosmetin and its glycosides, baicalin and its glycosides, santin and its glycosides, dinatin and its glycosides, nepitrin and its glycosides, or any combination thereof.
   45. The orally consumable composition of any one of clauses 1-44, wherein the orally consumable composition is a liquid composition.
   46. The orally consumable composition of any one of clauses 1-45, wherein the orally consumable composition is in the form of a beverage.
   47. The orally consumable composition any one of clauses 1-46, wherein the orally consumable composition is in the form of a carbonated beverage.
   48. The orally consumable composition of any one of clauses 1-46, wherein the orally consumable composition is in the form of a non-carbonated beverage.
   49. The orally consumable composition of any one of clauses 1-45, wherein the orally consumable composition is in the form of a liquid concentrate.
   50. The orally consumable composition of any one of clauses 1-44, wherein the orally consumable composition is in the form of a solid composition.
   51. The orally consumable composition of clauses 50, wherein the orally consumable composition is in the form of a powder, granule, capsule, tablet, compacted or compressed soluble solid form, food additive, or foodstuff.
   52. A method for providing a cognitive benefit to a human subject, comprising orally administering to the subject an orally consumable composition according to any one of clauses 1-51.
   53. The method of clause 52, wherein the cognitive benefit is selected from a group comprising improved performance, improved attention, improved memory, improved alertness, improved tranquility, reduced stress, reduced mental fatigue and any combination thereof.
   54. The method of clauses 52 or 53, wherein the method further comprises the step of conducting a cognitive assessment of the subject prior to and/or after the subject has consumed the orally consumable composition of any one of the clauses 1-5.

While certain embodiments have been illustrated and described, it should be understood that changes and modifications can be made therein in accordance with ordinary skill in the art without departing from the invention.

What is claimed is:

1. An orally consumable composition comprising:
(i) at least one aqueous extractive(s) from a plant; and/or
(ii) at least one essential oil(s) from the same or different plant; and/or
(iii) caffeine, wherein the caffeine is from a source other than (i) and/or (ii);
wherein (i) and/or (ii) form a plant extract composition; and wherein the ratio of total caffeine to plant extract composition is from 1:20 to 40:1 by weight (w/w).

2. The orally consumable composition of claim 1, wherein the at least one essential oil(s), comprise terpenes, monoterpenes, sesquiterpenes, diterpenes, esters, aldehydes, ketones, alcohols, phenols and oxides, and derivatives and/or combinations thereof.

3. The orally consumable composition of claim 2, wherein if both the essential oil(s) and the at least one aqueous extractive(s) are present, the ratio of the at least one essential oil(s) to the at least one aqueous extractive(s) is from 1:100 to 1:4.

4. The orally consumable composition of claim 3, wherein the at least one essential oils(s) comprise plant essential oil derived from the plant root, rhizomes, stem, bark, twig, leaves, flowers, fruits, seeds and combinations thereof.

5. The orally consumable composition of claim 4, wherein the plant extract composition is derived from a plant genus selected from a group comprising Salvia, Eucalyptus, Mentha, Thymus, Ocimum, Cinnamomum, Elettaria, Amomum, Pimpinella, Angelica, Zingiber, Piper, Petroselinum, Coriandrum, Foeniculum, Syzygium, Satureia, Camellia, Coffea, Theobroma, Iex, Paullinia, Euterpe, Ginkgo, Hibiscus, Melissa, Aspalathus, Rosa, Moringa, Magnolia, Olea, Sophora, Crocus, Hypericum, Polygonum, Citrus, Malus, Vitis, Prunus, Litchi, Vaccinium, Lycium, Fragaria, Punica, Ribes, Sambucus, Aronia, Foeniculum, Pimpinella, Beta, Curcuma, Glycine, Juglans, Murraya, Capsicum and combinations thereof.

6. The orally consumable composition of claim 5, wherein the plant extract composition comprises a Salvia species, wherein the Salvia species is selected from S. officinalis, S. lavandulifolia, S. rosmarinus (Rosmarinus officinalis), S. hispanica, S. columbariae, S. polystachya, S. bowleyana, S. cavaleriei, S. chinensis, S. flava, S. prionitis, S. sonchifolia, S. yunnanensis, S. miltiorrhiza, S. multicaulis, and S. divinorum.

7. The orally consumable composition of claim 5, wherein the plant extract composition comprises a Eucalyptus species, wherein the Eucalyptus species is selected from a group comprising Eucalyptus species is selected from E. globulus, E. cneorifolia, E. dives, E. Dumosa, E. goniocalyx, E. horistes, E. kochii, E. leucoxylon, E. largiflorens, E. oleosa, E. polybractea, E. radiata, E. rossii, E. sideroxylon, E. smithii, E. staigeriana, E. tereticornis, E. viridis and combinations thereof.

8. The orally consumable composition of claim 5, wherein the plant extract composition comprises a Mentha species, wherein the Mentha species is selected from a group comprising Mentha piperita, M. spicata, M. citrate and combinations thereof.

9. The orally consumable composition of claim 5, wherein the plant extract composition comprises a Thymus species, wherein the Thymus species is selected from a group comprising Thymus vulgaris, T. zygis, T. serpyllum, T. citriodorus, T. praecox, T. pseudolanuginosus and combinations thereof.

10. The orally consumable composition of claim 5, wherein the plant extract composition comprises a Ocimum species, wherein the Ocimum species is selected from a group comprising Ocimum basilicum, O. citriodorum, O. kilimandscharicum, O. Americanum, O. gratissimum, O. tenuiflorum and combinations thereof.

11. An orally consumable composition comprising:
(i) caffeine, wherein a total caffeine content is between 25 mg to 300 mg;
(ii) a plant extract composition comprising at least one aqueous extractive(s) from a plant and/or at least one essential oil(s) from the same or different plant, wherein the plant extract composition is between 1 mg to 500 mg;
wherein the plant extract composition is derived from plant genus Salvia.

12. The orally consumable composition of claim 11, comprising from 1 mg to 25 mg of the at least one essential oil(s).

13. The orally consumable composition of claim 12, wherein the at least one aqueous extractive(s) comprises: a rosmarinic acid and/or derivatives thereof in an amount of from 2.5 mg to 100 mg;
a carnosol, a carnosic acid, and/or derivatives thereof in an amount of from 0.5 mg to 100 mg;
caffeic acid and/or derivatives thereof in an amount of from 2.5 mg to 100 mg, and luteolin and/or derivatives thereof in an amount of from 2.5 mg to 100 mg, in a total amount of up to 500 mg,

14. The orally consumable composition of claim 12, wherein the plant extract composition is a S. officinalis extract composition that comprises rosmarinic acid in an amount of about 3.5 mg to 50 mg.

15. The orally consumable composition of claim 11, comprising about 150 mg of the plant extract composition, wherein the plant extract composition is a S. officinalis extract composition that comprises rosmarinic acid in an amount of about 7.8 mg, and total polyphenol in an amount of about 27 mg.

16. The orally consumable composition of claim 11, comprising about 250 mg of the plant extract composition, wherein the plant extract composition is a S. officinalis extract composition that comprises rosmarinic acid in an amount of about 12.9 mg, and total polyphenol in an amount of about 45 mg.

17. The orally consumable composition of claim 14, wherein the orally consumable composition comprises about 60-75 mg caffeine.

18. A method for providing a cognitive benefit to a human subject, comprising orally administering to the subject an orally consumable composition according to claim 17.

19. The method of claim 18, wherein the cognitive benefit is selected from a group comprising improved performance, improved attention, improved memory, improved alertness, improved tranquility, reduced stress, reduced mental fatigue and any combination thereof.

20. The method of claim 19, wherein the method further comprises the step of conducting a cognitive assessment of the subject prior to and/or after the subject has consumed the orally consumable composition of claim 17.
